(19) United States
(12) Patent Application Publication
(10) Pub. No.: US 2005/0233993 A1
(43) Pub. Date: Oct. 20, 2005

(54) Methods for promoting homologous recombination

(76) Inventors: James T. Kadonaga, Del Mar, CA (US); Vassilios Alexiadis, San Diego, CA (US)

Correspondence Address: FISH & RICHARDSON, PC, 12390 EL CAMINO REAL, SAN DIEGO, CA, 92130-2081, US

(21) Appl. No.: 10/516,982

(22) PCT Filed: Jan. 21, 2003
(86) PCT No.: PCT/US03/01787

§ 371 Date: Jun. 21, 2005

Related U.S. Application Data
(60) Provisional application No. 60/386,284, filed on Jun. 5, 2002.

(51) Int. Cl. A61K 48/00; C12N 15/85

(52) U.S. Cl. 514/44; 435/455

(57) ABSTRACT

Nucleosomal polynucleotides promote homologous strand pairing by recombinase. In the absence of superhelical tension, the efficiency of strand pairing with nucleosomal polynucleotides is substantially higher than that with non-nucleosomal nucleic acids. In addition, a recombinase comprising Rad54 and Rad51 associated activity can function cooperatively in the ATP-dependent remodeling of a nucleosomal polynucleotide (i.e., chromatin).

### CROSS REFERENCE TO RELATED APPLICATIONS

This application claims priority from U.S. Provisional Application Ser. No. 60/386,284, filed Jun. 5, 2002, which is incorporated herein by reference.

### STATEMENT AS TO FEDERALLY SPONSORED RESEARCH

This invention was made with government support under grant R01 GM 58272 awarded by the National Institutes of Health (NIH). The U.S. Government may have an interest in the subject matter of this patent application.

### FIELD OF THE INVENTION

The invention relates to methods for promoting homologous strand pairing and homologous recombination using nucleosomal polynucleotides.

### BACKGROUND

The process of recombination (homologous or site-specific) can generally be described as the exchange of nucleic acid segments anywhere along a length of two nucleic acid molecules. For example, a necessary step in homologous recombination is strand exchange, which involves a pairing of a nucleic acid duplex with at least one nucleic acid strand containing a complementary sequence to form an intermediate recombination structure containing heteroduplex nucleic acids. The heteroduplex nucleic acid may take several forms, including a three DNA strand containing triplex form wherein a single complementary strand invades the DNA duplex and, when two complementary DNA strands pair with a DNA duplex, a classical Holliday recombination joint or chi structure may form, or a double-D loop. Once formed, a heteroduplex structure may be resolved by strand breakage and exchange, so that all or a portion of an invading DNA strand is spliced into a recipient DNA duplex, adding or replacing a segment of the recipient DNA duplex. Alternatively, a heteroduplex structure may result in gene conversion, wherein a sequence of an invading strand is transferred to a recipient DNA duplex by repair of mismatched bases using the invading strand as a template. Whether by the mechanism of breakage and rejoining or by the mechanism(s) of gene conversion, formation of heteroduplex DNA at homologously paired joints can serve to transfer genetic sequence information from one DNA molecule to another.

Several proteins or purified extracts having the property of promoting homologous recombination (i.e., recombinase activity) have been identified in prokaryotes and eukaryotes (Cox and Lehman, Ann. Rev. Biochem. 56:229). These general recombinases promote one or more steps in the formation of homologously-paired intermediates, strand-exchange, gene conversion, and/or other steps in the process of homologous recombination.

The ability of eukaryotic cells to incorporate exogenous genetic material into genes residing on chromosomes has demonstrated that these cells have the general enzymatic machinery for carrying out homologous recombination required between resident and introduced sequences. Despite this ability, exogenous sequences transferred into eukaryotic cells undergo homologous recombination with homologous endogenous sequences only at very low frequencies. Thus, there exists a need in the art for methods that promote homologous pairing and homologous recombination.

### SUMMARY OF THE INVENTION

Methods for promoting homologous strand pairing between a nucleosomal polynucleotide and a target nucleic acid sequence are provided. Homologous strand pairing promotes homologous recombination by facilitating heteroduplex formation between the nucleosomal polynucleotide and the target nucleic acid sequence. The methods can be used to facilitate the transfer of specific polynucleotide sequences, in vitro or in vivo, to a target nucleic acid. The methods encompass, but are not limited to: 1) correcting or generating genetic mutations in endogenous DNA sequences by homologous recombination and/or gene conversion; 2) producing homologously targeted transgenic animals and plants at high efficiency, and 3) other applications (e.g., targeted drug delivery) based on enhanced in vitro or in vivo homologous strand pairing.

The invention generally provides methods for promoting homologous recombination by facilitating strand pairing between a nucleosomal polynucleotide and a target nucleic acid. The nucleosomal polynucleotide can comprise chemical substituents. Further, a recombinase can optionally be associated with the nucleosomal polynucleotide. Methods of the invention can be used in conjunction with compositions that comprise a nucleosomal polynucleotide and targeting or cell-uptake components to facilitate intracellular uptake of a nucleosomal polynucleotide, especially for in vivo gene therapy and gene modification. Accordingly, the present invention generally provides methods for targeting and altering, by homologous recombination, a target nucleic acid sequence in vitro or in vitro using a nucleosomal polynucleotide.

In one embodiment, a method for promoting homologous recombination between a nucleosomal polynucleotide and a target nucleic acid is provided. The method includes, but is not limited to, targeting at least one exogenous nucleosomal polynucleotide to a predetermined endogenous nucleic acid sequence and altering the endogenous nucleic acid sequence, such as a chromosomal DNA sequence, by homologous recombination within and/or flanking the targeted endogenous nucleic acid sequence. Optionally, the nucleosomal polynucleotide(s) may be introduced simultaneously or contemporaneously with a recombinase. Alternatively, the recombinase may be induced or produced in vivo, for example by expression of a heterologous expression cassette in a cell. The term “recombinase” includes polypeptides, or complexes thereof, capable of promoting strand exchange between a nucleosomal polynucleotide and a target nucleic acid. A recombinase can include, for example, Rad51 and Rad54 associated activity, or any homolog or functional equivalent thereof.

The invention further provides methods for correcting a genetic mutation in an endogenous target nucleic acid sequence by facilitating homologous strand pairing between a nucleosomal polynucleotide comprising a nucleic acid sequence that corrects or alters an endogenous target nucleic acid sequence. For example, the invention can be used to correct genetic mutations, such as base substitutions, additions, and/or deletions, by converting a mutant DNA sequence that encodes a non-functional, dysfunctional, and/or truncated polypeptide into a corrected DNA sequence that encodes a functional polypeptide (e.g., has a biological activity such as an enzymatic activity, hormone function, or other biological property). The methods and compositions of the invention may also be used to correct genetic mutations or dysfunctional alleles with genetic lesions in non-coding sequences (e.g., promoters, enhancers, silencers, origins of replication, splicing signals).

The invention also provides methods for inactivating gene expression by targeting a nucleic acid sequence with a nucleosomal polynucleotide that facilitates base substitution, addition, and/or deletion in a structural or regulatory endogenous DNA sequence to alter expression of one or more genes, typically by knocking out at least one allele of a gene (i.e., making a mutant, nonfunctional allele). The invention can also be used to correct disease alleles by producing a targeted alteration in the disease allele to correct a disease-causing lesion (e.g., a deletion).

Also provided are methods and compositions for diagnosis, treatment and prophylaxis of genetic diseases of animals, particularly mammals, wherein a nucleosomal polynucleotide and an optional recombinase are used to produce a targeted sequence modification in a disease allele of an endogenous gene. The invention may also be used to produce targeted sequence modification(s) in a non-human animal, particularly a non-human mammal such as a mouse, which create(s) a disease allele in a non-human animal. Sequence-modified non-human animals (i.e., transgenic animals) harboring such a disease allele may provide useful models of human and veterinary disease(s). Alternatively, the methods and compositions of the invention can be used to provide nonhuman animals having homologously-targeted human disease alleles integrated into a non-human genome; such non-human animals may provide useful experimental models of human or other animal genetic disease, including neoplastic and other pathogenic diseases.

Also provided are methods for enhanced homologous recombination of a nucleosomal polynucleotide to a targeted nucleic acid sequence in an endogenous chromosome to form a stable multistrand complex, and thereby alter expression of a predetermined gene sequence by interfering with transcription of sequence(s) adjacent to the multistrand complex. A recombinase which facilitates homologous strand pairing and stable multistrand complex formation is optionally included in the method. For example, a nucleosomal polynucleotide optionally associated with a recombinase may homologously pair with an endogenous chromosomal sequence in a structural or regulatory sequence of a gene and form a stable multistrand complex.

In addition, methods and compositions for treating or preventing acquired human and animal diseases, particularly parasitic or viral diseases by targeting viral gene sequences with a nucleosomal polynucleotide and thereby inactivating the viral gene sequences and inhibiting viral-induced pathology, are provided.

### BRIEF DESCRIPTION OF THE DRAWINGS

FIG. 1A illustrates isolated Drosophila Rad51 and Rad54 polypeptides.

FIG. 1B illustrates the formation of D-loops using isolated Drosophila Rad51 and Rad54 polypeptides.

FIG. 2A illustrates Micrococcal nuclease digestion analysis of chromatin reconstituted by salt dialysis.

FIG. 2B illustrates Rad51+Rad54 mediated D loop formation versus RecA mediated D-loop formation in the presence of naked DNA or salt dialysis chromatin (SD chromatin).

FIG. 2C illustrates the kinetics of D-loop formation with naked DNA and chromatin. Reactions were performed as in FIG. 2B, except that they were allowed to proceed for the indicated times after the addition of Rad54 and homologous DNA.

FIG. 3A illustrates relaxation of plasmid DNA and chromatin by topoisomerase I.

FIG. 3B illustrates D loop formation with relaxed chromatin. “++” indicates twice the topoisomerase I concentration than that used in the “+” lanes.

FIG. 4A illustrates ACF-mediated chromatin assembly.

FIG. 4B illustrates strand pairing reactions.

FIG. 5 illustrates restriction enzyme accessibility assays indicating that Rad54 and Rad51 function cooperatively in the remodeling of chromatin.

### DETAILED DESCRIPTION

Homologous strand pairing promotes homologous recombination by facilitating heteroduplex formation. The methods provided herein promote heteroduplex formation by utilizing a nucleosomal polynucleotide and optionally a recombinase comprising activity associated with Rad51 and Rad54. The chromatin-mediated process can be used to facilitate the transfer of specific polynucleotide sequences, in vitro or in vivo, to a target nucleic acid sequence.

For example, and not by way of any limitation, a list of the biological products which can be made by the present methods includes 1) a cloned nucleic acid (e.g., cDNA, partial or complete gene), 2) single or multiple cell lines each having a different targeted modification in one or more endogenous genes, 3) single or multiple cell lines having the insertion, substitution or deletion of one or more exogenous genes or modified genes, 4) transgenic animals each having cells which have a targeted modification of one or more endogenous alleles including disruption of gene function (knock-out) or modification of the gene product or its expression level as well as transgenic animals having exogenous nucleic acids incorporated into one or more cells.

The term “homologous” as used herein denotes a characteristic of a nucleic acid sequence, wherein a nucleic acid sequence has at least about 60 percent sequence identity as compared to a reference sequence, typically at least about 75 percent sequence identity, and preferably at least about 95 percent sequence identity as compared to a reference sequence. The percentage of sequence identity is calculated excluding small deletions or additions which total less than 25 percent of the reference sequence. The reference sequence may be a subset of a larger sequence, such as a portion of a gene or flanking sequence, or a repetitive portion of a chromosome. However, the reference sequence is at least 12-18 nucleotides long, typically at least about 30 nucleotides long, and preferably at least about 50 to 100 nucleotides long. In general, recombination efficiency increases with the length of the targeting polynucleotide portion that is substantially complementary to a reference sequence present in the target DNA.

As used herein, a “nucleosomal polynucleotide” includes any nucleic acid associated with histone core proteins, or histone-like core proteins, forming a chromatin-like structure. The nucleic acid can comprise, for example, a nucleotide sequence encoding a polypeptide (i.e., “gene”), or fragment thereof. A nucleosomal polynucleotide includes a sufficient number nucleotides covalently linked together to form at least one nucleosome with core histone polypeptides. Thus, the term “nucleosomal polynucleotide” includes chromatin minimally comprising core histones. The term “chromatin-associated,” as used herein, is equivalent to the term “nucleosomal polynucleotide-associated.”

A nucleosomal polynucleotide can include additional non-core histone proteins, such as H1 proteins, or additional non-histone proteins, such as HMG proteins. In general, a nucleosome core comprises an octamer of core histones (two each of H2A, H2B, H3 and H4) around which is wrapped a nucleic acid of approximately 150-200 base pairs. The nucleic acid is generally DNA. In addition, a linker DNA segment of approximately 50 base pairs can be associated with non-core histone H1 (or a related linker histone in certain specialized cells. In general, a nucleosomal polynucleotide may comprise any number of structures, as long as the changes do not substantially effect the functional ability of the polynucleotide to participate in homologous recombination.

Nucleosomal polynucleotides are generally at least about 2 to 100 nucleotides long, preferably at least about 5- to 100 nucleotides long, at least about 250 to 500 nucleotides long, more preferably at least about 500 to 2000 nucleotides long, or longer. The length of homology may be selected at the discretion of the practitioner on the basis of the sequence composition and complexity of the target nucleic acid sequence(s) and guidance provided in the art (Hasty et al., Molec. Cell. Biol. 11:5586, 1991; Shulman et al., Molec. Cell. Biol. 10:4466, 1990, which are incorporated herein by reference). Any length of homology sufficient to support nucleosomal polynucleotide associated homologous recombination is encompassed by the present methods.

Nucleosomal polynucleotides have at least one sequence that substantially corresponds to, or is substantially complementary to, a target nucleic acid sequence (i.e., a DNA sequence of a polynucleotide located in a target cell, target virus, or target plasmid, such as a chromosomal, mitochondrial, chloroplast, viral, episomal, or mycoplasmal polynucleotide). Such nucleosomal polynucleotide sequences serve as templates for homologous pairing with the predetermined target nucleic acid sequence(s). In the nucleosomal polynucleotides of the invention, such regions of homology are typically located at or near the 5′ or 3′ end (Berinstein et al., Molec. Cell. Biol. 12:360, 1992, which is incorporated herein by reference).

A nucleic acid may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine and hypoxathanine, etc. Thus, for example, chimeric DNA-RNA molecules may be used such as described in Cole-Strauss et al., Science 273:1386 (1996) and Yoon et al., PNAS USA 93:2071 (1996), both of which are hereby incorporated by reference.

As used herein, an “exogenous nucleosomal polynucleotide” is a polynucleotide which is transferred into a target cell but which has not been replicated in that host cell; for example, a virus genome polynucleotide that enters a cell by fusion of a virion to the cell is an exogenous polynucleotide, however, replicated copies of the viral polynucleotide subsequently made in the infected cell are endogenous sequences (and may, for example, become integrated into a cell chromosome). Similarly, transgenes which are microinjected or transfected into a cell are exogenous polynucleotides, however integrated and replicated copies of the transgene(s) are endogenous sequences.

A nucleic acid will generally contain phosphodiester bonds, although in some cases nucleic acid analogs are included that may have alternate backbones, comprising, for example, phosphoramide, phosphorothioate, phosphorodi-thioate, O-rmethylphophoroamidite linkages and peptide nucleic acid backbones and linkages. These modifications of the ribose-phosphate backbone or bases may be done to facilitate the addition of other moieties such as chemical constituents, including 2′ O-methyl and 5′ modified substituents or to increase the stability and half-life of such molecules in physiological environments.

As used herein, the term “target nucleic acid sequence” refers to polynucleotide sequences suitable for recombination with a nucleosomal polynucleotide. Such sequences include those contained in a target cell and include, for example, chromosomal sequences (e.g., structural genes, regulatory sequences including promoters and enhancers, recombinatorial hotspots, repeat sequences, integrated proviral sequences, hairpins, palindromes), episomal or extrachromosomal sequences (e.g., replicable plasmids or viral or parasitic replication intermediates) including chloroplast and mitochondrial DNA sequences. The target sequence may be selected at the discretion of the practitioner on the basis of known or predicted sequence information, and is not constrained to specific sites recognized by certain site-specific. In some embodiments, the target sequence will be other than a naturally occurring germline DNA sequence (e.g., a transgene, parasitic, mycoplasmal or viral sequence).

As used herein, “recombinase” refers to polypeptides having essentially all or most of the same functions, particularly the recombinase can: (i) properly bind to and position a nucleosomal polynucleotide to a homologous target and (ii) facilitate homologous recombination. In general, a recombinase facilitates intramolecular and intermolecular site specific recombination between specific sites in a nucleic acid sequence. A recombinase optionally included in a method of the invention generally comprises Rad51 and Rad54 associated activity and includes, for example, both yeast and mammalian Rad51 and Rad54 proteins, which mediate homologous pairing and strand-exchange reactions between ssDNA and homologous double-stranded DNA (Baumann et al., Cell 87:757; Gupta et al., Proc. Natl. Acad. Sci. USA 94:463; Sung, Science 265:241 (1994); Sung and Robberson Cell 82: 453, all incorporated herein by reference). It is understood that the term “Rad51 and Rad54 associated activity” specifically includes any Rad51 or Rad54 polypeptide from any species. The term “Rad51 and Rad54 associated activity” further includes homologues, derivatives, and fragments of Rad51 or Rad54. In a specific embodiment, a fusion protein containing Rad51 or Rad54 or a homologue, derivative, or fragment thereof may also be used in the recombinase. The fusion protein can include an epitope tag, such as glutathione-S-transferase (GST), c-myc, 6-histidine (6×-His), FLAG®, green fluorescent protein (GFP), maltose binding protein (MBP), influenza A virus haemagglutinin (HA), β-galactosidase, and GAL4.

Rad51 is a member of the RAD52 epistasis group of genes (which include RAD50, RAD52, RAD54, RAD55, RAD57, RAD59, MRE11 and XRS2) as components of the yeast recombinational repair pathway (Cromie et al., Mol. Cell 8:1163; Petrini et al., Semin. Immunol. 9:181). These genes are conserved from yeast to humans. Proteins known to interact with Rad51, including Rad52, Rad54, Rad55, DMC1, BRCA1, BRCA2, p53, UBC9, RNA polymerase II, and Rad51 itself. Both Rad51 and RecA are able to mediate strand invasion and annealing to yield a D loop, which is a key step in the recombination process. In this reaction, Rad51 (or RecA) forms a nucleoprotein filament on single-stranded DNA in the presence of ATP, and this filament is used for homologous pairing with a double-stranded DNA molecule. The efficiency of strand pairing by Rad51 can be stimulated by the presence of additional factors such as RP-A, the Rad55-Rad57 heterodimer, Rad52 and Rad54.

In addition to Rad51, Rad57 and Dmc1 share sequence homology with the recA gene. Phylogenetic analysis by Ogawa and co-workers suggest the existence of two sub-families within eukaryotic RecA homologs: the Rad51-like (Rad51 of human, mouse, chicken, S. cerevisiae, S. pombe and Mei3 of Neurospora crassa) and the Dmc1-like genes (S. cerevisiae Dmc1 and Lilium longiflorum LIM15). All these Rad51 genes share significant homology with residues 33-240 of the E. coli RecA protein, which have been identified as a “homologous core” region.

Rad54 is a member of the Snf2-like family of ATPases. The Snf2-like family includes proteins such as Swi2/Snf2, Sthl, ISWI, Ino80, and Mi-2/CHD3/CHD4, which are the ATPase subunits of chromatin remodeling factors that catalyze the mobilization of nucleosomes (see, for example: Fyodorov and Kadonaga, Cell 106:523).

Examples of additional recombinase proteins include, but are not limited to: RecA803, uvsX, and other RecA mutants and RecA-like recombinases (Roca, A. I., Crit. Rev. Biochem. Molec. Biol. 25:415), sep1 (Kolodner et al., Proc. Natl. Acad. Sci. (U.S.A.) 84:5560), RuvC (Dunderdale et al., Nature 354:506), DST2, KEM1, XRN1 (Dykstra et al., Molec. Cell. Biol. 11:2583), STPα/DST1 (Clark et al., Molec. Cell. Biol. 11:2576), and HPP-1 (Moore et al., Proc. Natl. Acad. Sci. (U.S.A.) 88:9067).

It is understood that a recombinase may actually be a complex of proteins, i.e. a “recombinosome”. Included within the definition of a recombinase are portions or fragments of recombinases which retain recombinase biological activity, as well as variants or mutants of wild-type recombinases which retain biological activity.

Recombinase mediated processes are further described in the following publications WO 00/63365, WO 99/60108, WO 00/56872, WO 99/37755, U.S. Pat. Nos. 5,948,653, 6,074,853, 5,763,240, 5,929,043, 5,989,879, and U.S. Ser. No. 09/654,108, all of which are incorporated herein in their entirety by reference.

The present invention provides enhanced strand pairing and homologous recombination using nucleosomal polynucleotides. Examples of nucleosomal polynucleotide mediated processes include, without limitation, the use of nucleosomal polynucleotides to form single- or double-D loops with homologous target nucleic acid sequences to either isolate the homologous nucleic acid or to facilitate enhanced homologous reombination in vitro or in vivo. Thus, the invention provides methods for promoting homologous recombination by using a nucleosomal polynucleotide comprising histones and contacting the polynucleotide with a target nucleic acid sequence under conditions that support homologous recombination. As used herein, “contacting” encompasses in vitro and in vivo contacting to facilitate recombination.

The present invention provides methods for targeting and altering, by homologous recombination, a target nucleic acid sequence in a target cell, to make targeted sequence modifications. The methods comprise introducing into the target cells nucleosomal polynucleotides at least partially or substantially complementary to a target nucleic acid sequence. Subsequently, the target cells can be screened to identify target cells containing the targeted sequence modification.

The methods provided herein can be used to modify a disease allele such that the disease phenotype resulting from the disease allele is altered to a non-disease state. As used herein, the term “disease allele” refers to an allele of a gene which is capable of producing a recognizable disease. A disease allele may be dominant or recessive and may produce disease directly or when present in combination with a specific genetic background or pre-existing pathological condition. A disease allele may be present in the gene pool or may be generated de novo in an individual by somatic mutation.

Suitable endogenous target nucleic acids include, but are not limited to, nucleic acids comprising genes which encode peptides or proteins including enzymes, structural or soluble proteins, as well as endogeneous regulatory sequences including, but not limited to, promoters, transcriptional or translational sequences and repetitive sequences. Additional example include genes which encode lactoglobulins including both α-lactoglobulin and β-lactoglobulin; casein, including both α-casein, β-casein and κ-casein; albumins, including serum albumin, particularly human and bovine; immunoglobulins, including IgE, IgM, IgG and IgD and monoclonal antibodies; globin; integrin; hormones; growth factors, particularly bovine and human growth factors, including transforming growth factor, epidermal growth factor, nerve growth factors, etc.; collagen; interleukins, including IL-1 to IL-17; a major histocompatibility antigen (MHC); G-protein coupled receptors (GPCR); nuclear receptors; ion channels; multidrug resistance genes; amyloid proteins; enzymes, including esterases, proteases (including tissue plasminogen activator (tPA)), lipases, carbohydrases, etc.; APRT, HPRT; leptin; tumor suppressor genes; provirus; prions; OTC; CFTR; sugar transferases such as alpha-galactosyl transferase (galT) or fucosyl transferase; a milk or urine protein gene including the caseins, lactoferrin and whey proteins; oncogenes; cytokines, particularly human; transcription factors; and other pharmaceuticals. Any or all of these may also be suitable exogeneous genes to be add to a genome using the methods outlined herein.

In one aspect, nucleosomal polynucleotides are optionally associated with a recombinase. The recombinase can include Rad51 and Rad54 associated activities. The conditions used to coat polynucleotides with recombinases such as a Rad51 recombinase have been described in U.S. Pat. No. 5,273,881, which is incorporated herein by reference.

Alternatively, a recombinase (prokaryotic, eukaryotic or endogeneous to the target cell) may be exogenously induced or administered to a target cell simultaneously or contemporaneously (i.e., within about a few hours) with the targeting polynucleotide(s). Such administration is typically done by micro-injection, although electroporation, lipofection, and other transfection methods known in the art may also be used.

Alternatively, a recombinase may be produced in vivo. For example, they may be produced from a homologous or heterologous expression cassette in a transfected cell or transgenic cell, such as a transgenic totipotent cell (e.g. a fertilized zygote) or an embryonal stem cell (e.g., a murine ES cell such as AB-1) used to generate a transgenic non-human animal line or a somatic cell or a pluripotent hematopoietic stem cell for reconstituting all or part of a particular stem cell population (e.g. hematopoietic) of an individual. Conveniently, a heterologous expression cassette includes a modulatable promoter, such as an ecdysone-inducible promoter-enhancer combination, an estrogen-induced promoter-enhancer combination, a CMV promoter-enhancer, an insulin gene promoter, or other cell-type specific, developmental stage-specific, hormone-inducible, or other modulatable promoter construct so that expression of at least one species of recombinase from the cassette can by modulated for transiently producing recombinase(s) in vivo simultaneous or contemporaneous with introduction of a nucleosomal polynucleotide into the cell. When a hormone-inducible promoter-enhancer combination is used, the cell must have the required hormone receptor present, either naturally or as a consequence of expression a co-transfected expression vector encoding such receptor.

Alternatively, the recombinase may be endogeneous and produced in high levels. In this embodiment, preferably in eukaryotic target cells such as tumor cells, the target cells produce an elevated level of recombinase. In other embodiments the level of recombinase may be induced by DNA damaging agents, such as mitomycin C, UV or .gamma.-irradiation. Alternatively, recombinase levels may also be elevated by transfection of a virus or plasmid encoding the recombinase gene into the cell.

In another embodiment, exogenous nucleosomal polynucleotides can be used to inactivate, decrease or alter the biological activity of one or more genes in a cell (or transgenic nonhuman animal or plant). This finds particular use in the generation of animal models of disease states, or in the elucidation of gene function and activity, similar to “knock out” experiments. Alternatively, the biological activity of the wild-type gene may be either decreased, or the wild-type activity altered to mimic disease states. This includes genetic manipulation of non-coding gene sequences that affect the transcription of genes, including, promoters, repressors, enhancers and transcriptional activating sequences.

For making transgenic nonhuman animals (which include homologously targeted non-human animals) embryonal stem cells (ES cells) and fertilized zygotes are preferred. In a preferred embodiment, embryonal stem cells are used. Murine ES cells, such as AB-1 line grown on mitotically inactive SNL76/7 cell feeder layers (McMahon and Bradley, Cell 62: 1073-1085 (1990)) essentially as described (Robertson, E. J. (1987) in Teratocarcinomas and Embrvonic Stem Cells: A Practical Approach. E. J. Robertson, ed. (oxford: IRL Press), p. 71-112) may be used for homologous gene targeting. Other suitable ES lines include, but are not limited to, the E14 line (Hooper et al. (1987) Nature 326: 292-295), the D3 line (Doetschman et al. (1985) J. Embryol. Exp. Morph. 87: 21-45), and the CCE line (Robertson et al. (1986) Nature 323: 445-448). The success of generating a mouse line from ES cells bearing a specific targeted mutation depends on the pluripotence of the ES cells (i.e., their ability, once injected into a host blastocyst, to participate in embryogenesis and contribute to the germ cells of the resulting animal).

The pluripotence of any given ES cell line can vary with time in culture and the care with which it has been handled. The only definitive assay for pluripotence is to determine whether the specific population of ES cells to be used for targeting can give rise to chimeras capable of germline transmission of the ES genome. For this reason, prior to introduction of a nucleosomic polynucleotide, a portion of the parental population of AB-1 cells is injected into C57B1/6J blastocysts to ascertain whether the cells are capable of generating chimeric mice with extensive ES cell contribution and whether the majority of these chimeras can transmit the ES genome to progeny.

In addition, non-human zygotes can be used, for example to make transgenic animals, using techniques known in the art (see U.S. Pat. No. 4,873,191). Preferred zygotes include, but are not limited to, animal zygotes, including fish, avian and mammalian zygotes. Suitable fish zygotes include, but are not limited to, those from species of salmon, trout, tuna, carp, flounder, halibut, swordfish, cod, tulapia and zebrafish. Suitable bird zygotes include, but are not limited to, those of chickens, ducks, quail, pheasant, turkeys, and other jungle fowl and game birds. Suitable mammalian zygotes include, but are not limited to, cells from horses, cattle, buffalo, deer, sheep, rabbits, rodents such as mice, rats, hamsters and guinea pigs, goats, pigs, primates, and marine mammals including dolphins and whales. See Hogan et al., Manipulating the Mouse Embryo (A Laboratory Manual), 2nd Ed. Cold Spring Harbor Press, 1994, incorporated by reference.

In general, transgenic animals are made with any number of changes. Exogeneous nucleosomal polynucleotide sequences, or extra copies of endogeneous sequences, including structural genes and regulatory sequences, may be added to the animal. Endogeneous sequences (again, either genes or regulatory sequences) may be disrupted, i.e. via insertion, deletion or substitution, to prevent expression of endogeneous proteins. Alternatively, endogeneous sequences may be modified to alter their biological function, for example via mutation of the endogeneous sequence by insertion, deletion or substitution using a method of the invention.

The methods of the present invention are useful to add exogenous DNA sequences, such as exogenous genes or regulatory sequences, extra copies of endogenous genes or regulatory sequences, or exogeneous genes or regulatory sequences, to a transgenic plant or animal. This may be done for a number of reasons: for example, adding one or more copies of a wild-type gene can increase the production of a desirable gene product; adding or deleting one or more copies of a therapeutic gene can alleviate a disease state, or to create an animal model of disease. Adding one or more copies of a modified wild type gene may be done for the same reasons. Adding therapeutic genes or proteins may yield superior transgenic animals, for example for the production of therapeutic or nutriceutical proteins. Adding human genes to non-human mammals may facilitate production of human proteins and adding regulatory sequences derived from human or non-human mammals may be useful to increase or decrease the expression of endogenous or exogenous genes. Such inserted genes may be under the control of endogenous or exogenous regulatory sequences, as described herein.

An endogenous target nucleic acid may be disrupted in a variety of ways by a homologous nucleosomal polynucleotide. The term “disrupt” as used herein comprises a change in the coding or non-coding sequence of an endogenous nucleic acid that alters the transcription or translation of an endogenous gene. In a preferred embodiment, a disrupted gene will no longer produce a functional gene product. Generally, disruption may occur by either the insertion, deletion or frame shifting of nucleotides.

Delivery of the nucleosomal polynucleotide into a target cell is the first critical step in the production of a transgenic non-human animal or in the use of gene therapy to treat a disease. A variety of methods for delivering and expressing a nucleic acid within a mammalian cell are known to those of ordinary skill in the art. Such methods include, for example liposome-based gene delivery (WO 93/24640; Mannino, BioTechniques 6:682, 1988; U.S. Pat. No. 5,279,833; WO 91/06309; Felgner et al., Proc. Natl. Acad. Sci. USA 84:7413, 1987; and Budker et al., Nature Biotechnology, 14:760, 1996). Other methods known to the skilled artisan include electroporation (U.S. Pat. Nos. 5,545,130, 4,970,154, 5,098,843, and 5,128,257), direct gene transfer, cell fusion, precipitation methods, particle bombardment, and receptor-mediated uptake (U.S. Pat. Nos. 5,547,932, 5,525,503, 5,547,932, and 5,460,831).

Ex vivo cell transfection for diagnostics, research, or for gene therapy (e.g., via re-infusion of the transfected cells into the host organism) is well known to those of skill in the art. Cells can be isolated from the subject organism, contacted with a nucleosomal polynucleotide, and re-infused back into the subject organism (e.g., patient). Various cell types suitable for ex vivo transfection are well known to those of skill in the art (see, e.g., Freshney et al., Culture of Animal Cells, A Manual of Basic Technique, third edition (1994)) and the references cited therein for a discussion of how to isolate and culture cells from patients). For example, stem cells can be used in ex-vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34⁺ cells in vitro into clinically important immune cell types using cytokines such a GM-CSF, IFN-γ and TNF-α are known (see, Inaba et al., J. Exp. Med. 176:1693, 1992). For example, in mice, stem cells are isolated from bone marrow cells by panning the bone marrow cells with antibodies which bind unwanted cells, such as CD4⁺ and CD8⁺ (T cells), CD45⁺, GR-1 (granulocytes) and differentiated antigen presenting cells. Human hematopoietic progenitor and stem cells are characterized by the presence of a CD34 surface membrane antigen. This antigen is used for purification, e.g., on affinity columns which bind CD34 (Ho et al., Stem Cells 13 (suppl. 3):100, 1995. Hematopoietic stem cells can also be isolated from fetal cord blood (Yu et al., Proc. Natl. Acad. Sci. USA, 92:699, 1995).

For in vivo transfection, compositions (e.g., liposomes) containing therapeutic nucleosomal polynucleotides can be administered directly to the organism for transduction of cells in vivo. Administration is by any of the routes suitable for introducing chromatin into target cells in vivo. The nucleosomal polynucleotides are administered in any suitable manner, preferably with pharmaceutically acceptable carriers. Suitable methods of administering such packaged nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition comprising a nucleosomal polynucleotide. Accordingly, there is a wide variety of suitable formulations of pharmaceutical compositions that can comprise nucleosomal polynucleotides for use in the methods of the present invention. For example, the packaged nucleosomal polynucleotides, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions comprising a nucleosomal polynucleotide and optionally a recombinase activity can be administered, for example, by intravenous infusion, orally, topically, intraperitoneally, intravesically or intrathecally.

Cells transduced by the nucleosomal polynucleotide as described above in the context of ex vivo therapy can also be administered intravenously or parenterally as described above. For administration, transduced cells can be administered at a rate determined by the LD-50 of the transduced cell type, and the side-effects of the cell type at various concentrations, as applied to the mass and overall health of the patient. Administration can be accomplished via single or divided doses.

In another embodiment, kits are provided. The kits can be utilized in a variety of applications, including the conversion of non-nucleosomal polynucleotides into nucleosomal polynucleotides and the transfer of the converted polynucleotide in to a suitable host cell. The kits can optionally include a recombinasea such as a recombinase comprising Rad51 and Rad54 associated activity. The kits can further include a means for detecting a polynucleotide that has recombined with a target nucleic acid sequence. For example, techniques such as PCR can be used to determine the presence or absence of a polynucleotide in a target sequence. Additional techniques are known to those skilled in the art. Additional reagents can also be included. Furthermore, the kit may include packaging and instructions, as required.

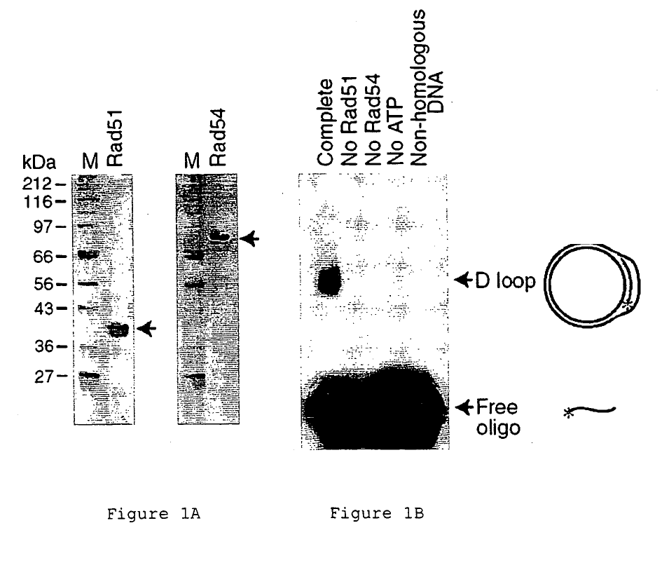

As shown in FIG. 1B, isolated, recombinantly produced Drosophila Rad51 and Rad54 catalyzed the formation of D loops in a manner that is dependent on the presence of homologous plasmid DNA (FIG. 1B). This process is substantially enhanced in the presence of plasmid DNA comprising nucleosomes.

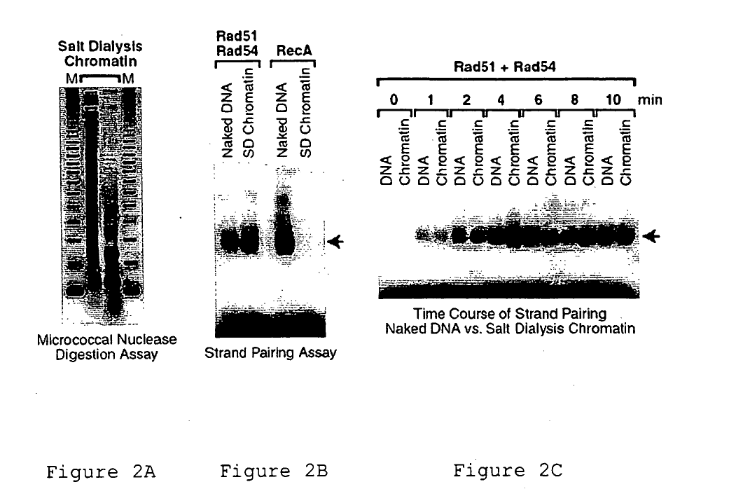

Chromatin-mediated (i.e., nucleosomal polynucleotide-mediated) D loop formation is demonstrated in FIG. 2B. Chromatin were reconstituted chromatin by salt dialysis techniques. The salt dialysis chromatin (SD chromatin) was prepared by gradually decreasing the salt concentration in a mixture of plasmid DNA and purified core histones from Drosophila embryos, and fully-reconstituted chromatin was separated from partially-reconstituted chromatin by sucrose gradient sedimentation. Micrococcal nuclease digestion analysis of the chromatin samples revealed that the salt dialysis chromatin consisted of closely-packed arrays of nucleosomes (see FIG. 2A). D loop formation occurred at a higher efficiency in the presence of nucleosomal polynucleotides than in the presence of non-nucleosomal DNA (FIG. 2B). Moreover, the rate of D loop formation by a recombinase comprising Rad51 and Rad54 associated activity with chromatin is similar to that seen with non-nucleosomal DNA (FIG. 2C). In contrast, the E. coli RecA protein is able to mediate D loop formation with naked DNA, but not with chromatin (FIG. 2B).

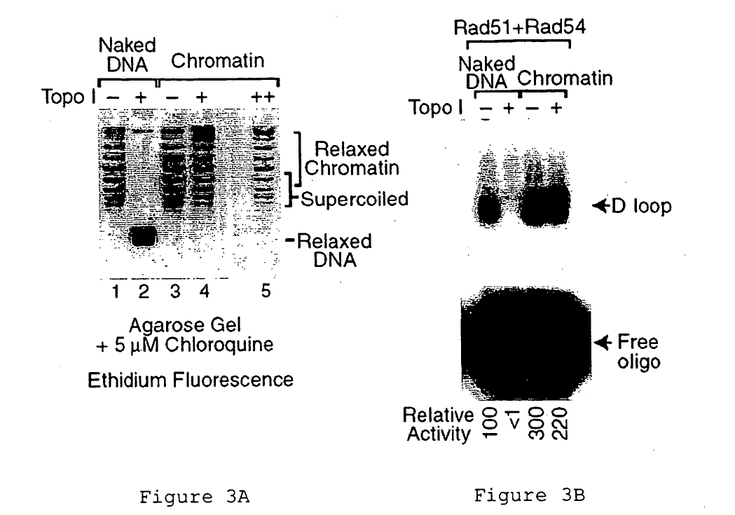

The data further indicates that, in the absence of superhelical tension, strand pairing in the presene of nucleosomal polynucleotides occurs with higher efficiency than in the presence of non-nucleosomal polynucleotides (see FIG. 3B, compare lanes 2 and 4). FIG. 3A shows chromatin treated with purified topoisomerase I. The chromatin was reconstituted by using supercoiled plasmid DNA in the absence of topoisomerases. Under these conditions, the DNA remains chemically unchanged, as no phosphodiester bonds are broken. In the absence of topoisomerase I, the number of supercoils in the non-nucleosomal DNA and chromatin are essentially identical (see FIG. 3A, compare lanes 1 and 3). When topoisomerase I is added to the chromatin, the unconstrained supercoils are relaxed, but upon deproteinization, the resulting DNA exhibits supercoils that are due to the wrapping of the DNA in nucleosomes (because the wrapping of the DNA around each histone octamer constrains approximately one negative supercoil; Germond et al., Proc. Natl. Acad. Sci. USA 72:1843) (FIG. 3A, compare lanes 3, 4, and 5).

Strand pairing reactions were performed with DNA and salt dialysis chromatin in the absence or presence of topoisomerase I (FIG. 3B). With non-nucleosomal DNA, a greater than 100-fold reduction in the efficiency of D loop formation upon relaxation of the template with topoisomerase I was observed (FIG. 3B, compare lanes 1 and 2). In contrast to the effects seen with naked DNA, relaxation of the chromatin by topoisomerase I has little effect upon the efficiency of D loop formation by Rad51 and Rad54. Thus, in the absence of superhelical tension, strand pairing by a recombinase comprising Rad51 and Rad54 associate activity occurs with higher efficiency in chromatin than in naked DNA (FIG. 3B, compare lanes 2 and 4).

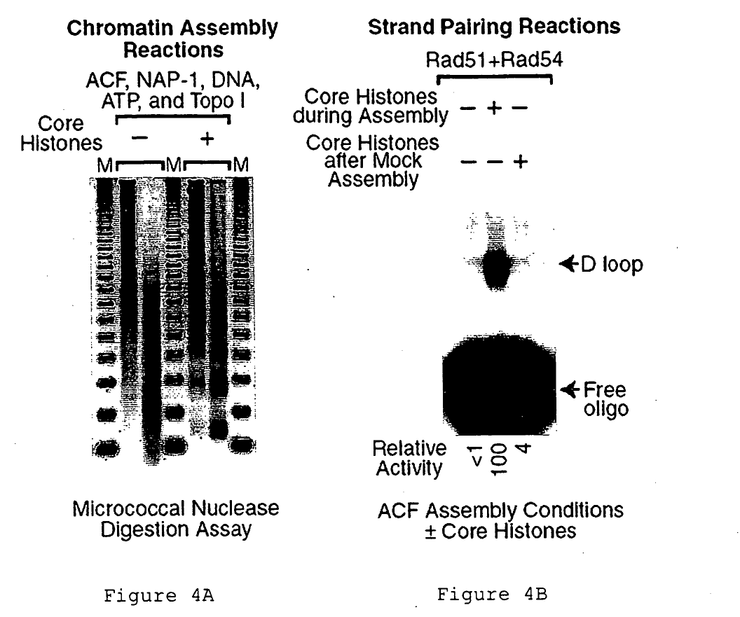

The effect of chromatin assembly upon the efficiency of D loop formation is shown in FIG. 4. Nucleosomal polynucleotides were assembled by using purified recombinant ACF, purified recombinant NAP-1, purified core histones, relaxed plasmid DNA, and ATP in the presence of purified topoisomerase I. The assembly reaction products were analyzed by micrococcal nuclease digestion (FIG. 4A). These samples were used as substrates for strand pairing by a recombinase comprising Rad51 and Rad54 associated activities. The addition of core histones during chromatin assembly results in a greater than 100-fold enhancement of strand pairing (FIG. 4B, compare left versus center lanes). In contrast, the addition of core histones after a mock assembly reaction (carried out in the absence of histones) did not stimulate D loop formation (FIG. 4B, right lane). These results indicate that strand pairing is enhanced by chromatin (i.e., nucleosomal polynucleotides) but not non-nucleosomal core histones as the packaging of relaxed DNA into chromatin results in a >100-fold stimulation of D loop formation by Rad54.

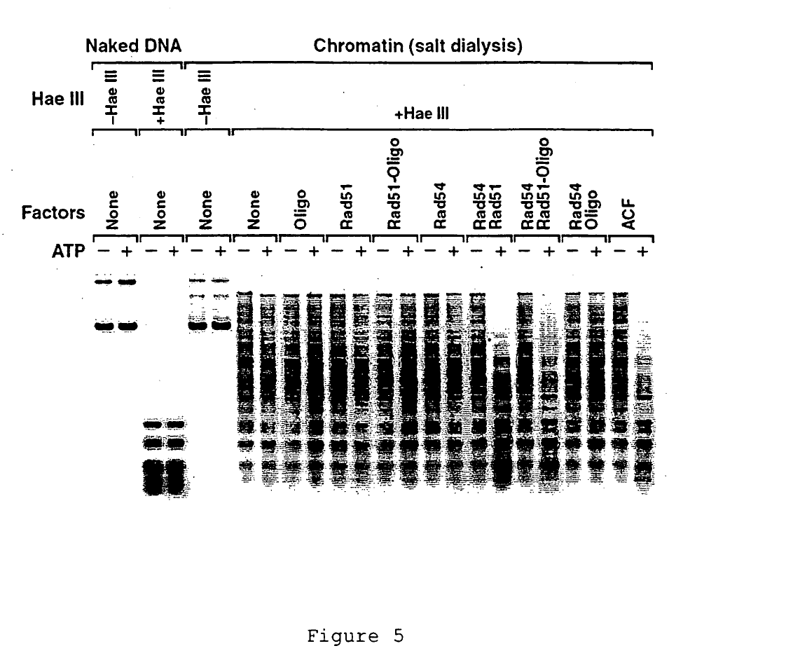

The invention further provides methods for chromatin modeling using a recombinase comprising at least Rad51 and Rad54 associated activity. FIG. 5 demonstrates the ability of Rad54 and/or Rad51 to facilitate the access of a restriction enzyme (Hae III) to DNA packaged into nucleosome arrays. ATP-utilizing chromatin assembly and remodeling factor (ACF) was used as a positive control. ACF acts catalytically to mediate the ATP-dependent assembly of histones (provided by histone chaperone proteins) into nucleosomes. This type of restriction enzyme accessibility assay has been used for the analysis of chromatin remodeling in vivo (Almer et al., EMBO J. 5:2689), the biochemical purification of the Chromatin assembly complex (CHRAC) chromatin remodeling factor (Varga-Weisz et al., Nature 388:598), the characterization of the INO80.com remodeling complex (Shen et al., Nature 406:541), and the comparative analysis of six chromatin remodeling complexes (ySWI/SNF, yRSC, hSWI/SNF, xMi-2, dCHRAC, dNURF; Boyer et al., J. Biol. Chem. 275:18864). As shown in FIG. 5, neither Rad54 alone nor Rad51 alone exhibited any detectable chromatin remodeling activity in the absence or presence of the DL2 oligonucleotide. In sharp contrast, Rad54 and Rad51 function cooperatively in the ATP-dependent remodeling of chromatin. The ability of a recombinase comprising Rad54 and Rad51 associated activity to rearrange chromatin structure is consistent with their ability to catalyze strand pairing with chromatin.

### Materials and Methods

Full-length cDNA clones that encode Drosophila Rad51 and Rad54 were obtained from Research Genetics and were subcloned into pFastBac1 (Gibco-BRL). Sequences that encode the FLAG epitope tag (DYKDDDDK (SEQ ID NO:2)) were introduced into both constructs at the 3′ end of the coding sequences. The Drosophila homolog of RAD54 has been termed okra and DmRAD54 (Kooistra et al., Mol. Cell. Biol. 19:6269). In this study, we refer to Drosophila Rad54 protein as Rad54.

Recombinant ACF, recombinant NAP-1, and core histones from Drosophila embryos were purified as previously described (Bulger and Kadonaga, Methods. Mol. Genet. 5:241). Rad51 and Rad54 proteins containing C-terminal FLAG tags were synthesized in S. frugiperda (Sf9) cells. The proteins were affinity-purified essentially as described for FLAG-tagged ACF (Ito et al., Genes Dev. 13:1529), with the following modifications. After incubation of the cell lysate with FLAG M2 resin (Sigma), the resin was washed four times with 12 ml each of wash buffer A (20 mM Tris-HCl, pH 7.9, 150 mM NaCl, 2 mM MgCl₂, 0.2 mM EDTA, 1 mM DTT, 15% (vol/vol) glycerol, 0.01% (vol/vol) NP-40, 10 mM α-glycerophosphate, 0.2 mM PMSF, 0.5 mM benzamidine-HCl, 2 μg/ml leupeptin, 1 μg/ml aprotinin) and two times with 12 ml each of wash buffer B (20 mM Hepes (K⁺), pH 7.6, 50 mM potassium glutamate, 0.2 mM EDTA, 15% (vol/vol) glycerol, 0.01% (vol/vol) NP-40, 1 mM DTT, 10 mM α-glycerophosphate, 0.1 mM PMSF, 0.5 mM benzamidine-HCl, 2 μg/ml leupeptin, 1 μg/ml aprotinin). The protein was eluted by four successive cycles of addition and removal of 100 μl elution buffer (wash buffer B containing 0.4 mg/ml FLAG peptide (Sigma) and 0.5 mg/ml recombinant human insulin (Roche)). As shown in FIG. 1A, FLAG-tagged Drosophila Rad51 and Rad54 were synthesized in Sf9 cells by using a baculovirus expression vector and affinity-purified with monoclonal antibodies that recognize the FLAG epitope. The proteins were subjected to 10% polyacrylamide-SDS gel electrophoresis. The proteins were visualized by staining with Coomassie Brilliant Blue R-250. Protein concentrations were estimated by polyacrylamide-SDS gel electrophoresis along with BSA standards.

RecA-His₆ protein, which contains a C-terminal His₆ tag, was synthesized in E. coli and purified by Ni(II) affinity chromatography under native conditions as described (QIA Expressionist, Qiagen), except that protein was eluted in the following buffer: 50 mM sodium phosphate, pH 8.0, 100 mM NaCl, 250 mM imidazole, 1 mM benzamidine, 1 mM PMSF, 10 mM 2-mercaptoethanol, 2.5 μg/ml aprotinin, 2.5 μg/ml pepstatin, and 2.5 μg/ml leupeptin. Commercially available RecA (Promega) was also used, and yielded identical results as those seen with the His₆-tagged RecA.

The pU6LNS plasmid (3291 bp; Pazin et al., Science 276:809) was purified by two successive CsCl isopycnic centrifugation steps. The 135-mer oligonucleotide DL2 (5′ GCA GTT CCC CTG CAT AAG GAT GAA CCG TTT TAC AAA GAG AAG CTT AAC TGC AAA ATT GGG CCA AAA TTG GGT CGG ATC CAT GGA AAT AAC ATA TGT GTA TCT TTA TCT TCC TGT ATG ATA TAG ATA ACT AAC ATC 3′ (SEQ ID NO:1)) is complementary to pU6LNS. The non-homologous DNA control used in FIG. 1 is a pFastBac1 derivative, and was also purified by two successive CsCl isopycnic centrifugation steps. The DL2 oligonucleotide was radiolabelled by incubation with T4 polynucleotide kinase (Promega) and [γ-³²P]ATP (ICN).

D loop reactions were performed essentially as described previously (Mazin et al., Mol. Cell 6:583). In a standard reaction, Rad51 was incubated with radiolabelled DL2 oligonucleotide in buffered medium [25 mM Tris-acetate, pH 7.5, 10 mM magnesium acetate, 100 μg/ml bovine serum albumin, 1 mM DTT, 2 mM ATP, 3 mM phosphoenolpyruvate, 20 units/ml pyruvate kinase] for 20 min at 27° C. Then, Rad54 and pU6LNS (as plasmid DNA or chromatin) was added, and the mixture was incubated for 4 min (unless stated otherwise) at 27° C. The reaction was terminated by the addition of EDTA to 50 mM and SDS to 1% (wt/vol). The sample was treated with proteinase K (500 μg/ml) for 10 min at 37° C., and then 1/10 volume of 20% Ficoll, 0.1% bromphenol blue was added. The resulting DNA species were resolved by 1% agarose gel electrophoresis, and the dried gel was subjected to autoradiography (see FIG. 1B).

The final concentrations of the D loop reaction components were as follows: Rad51 (200 nM), Rad54 (46 nM), ATP (2 mM), DL2 oligonucleotide (1 nM), and pU6LNS (4 nM). Under these conditions with chromatin templates (with excess chromatin relative to oligonucleotide), approximately 7% of the radiolabelled oligonucleotide is incorporated into the D loop.

Reactions with RecA were performed in analogous manner by incubation of purified E. coli RecA with radiolabelled DL2 oligonucleotide for 20 min at 27° C., followed by the addition of plasmid DNA and incubation for an additional 20 min at 27° C. The final concentration of RecA protein was 870 nM.

The ATP-dependent assembly of chromatin (i.e., nucleosomal polynucleotide) by purified recombinant ACF and NAP-1 was carried out under conditions known to the skilled artisan. Chromatin was reconstituted by salt dialysis with purified Drosophila core histones and plasmid DNA, and the resulting minichromosomes were purified by 15% to 50% sucrose gradient sedimentation (Jeong et al., J. Mol. Biol. 222:1131). As shown in FIG. 2A, the samples were subjected to partial digestion with two different concentrations of micrococcal nuclease and subsequently deproteinized. The resulting DNA fragments were resolved by agarose gel electrophoresis and visualized by staining with ethidium bromide. The mass markers (M) are the 123 bp DNA ladder (Gibco-BRL).

FIG. 2B compares the ability of Rad51+Rad54 versus RecA to mediate D loop formation with either naked DNA (i.e., non-nucleosomal polynucleotide) or a nucleosomal polynucleotide. Reactions were performed as in FIG. 1 except that the final concentration of Rad54 was 28 nM and that of DNA or chromatin was 1 nM. Reactions with RecA were performed in an analogous manner by incubation of purified E. coli RecA with radiolabelled DL2 oligonucleotide for 20 min at 27° C., followed by the addition of plasmid DNA and incubation for an additional 20 min at 27° C. The final concentration of RecA protein was 870 nM. The data indicate that Rad51 and Rad54, but not RecA, are able to mediate D loop formation with chromatin.

FIG. 2C shows the kinetics of D loop formation with naked DNA and chromatin. Reactions were performed as described in FIG. 2B except that they were allowed to proceed for the indicated times after the addition of Rad54 and homologous DNA.

The data provided herein further indicates that, in contrast to naked DNA, polynucleotides associated with core histone proteins enhance D loop formation by Rad51 and Rad54 in the absence of superhelical tension. As shown in FIG. 3A, plasmid DNA and chromatin (reconstituted by salt dialysis) were relaxed with purified recombinant Drosophila topoisomerase I (catalytic fragment). An aliquot of each of the samples was deproteinized and subjected to 1% agarose gel electrophoresis in the presence of 5 μM chloroquine followed by staining with ethidium bromide. As shown in FIG. 3B, the presence of relaxed chromatin in the reaction promotes D loop formation.

The data further indicates that the packaging of relaxed DNA into chromatin facilitates strand pairing by Rad51-associated and Rad54-associated activities. For example, FIG. 4A shows the results of chromatin assembly reactions performed with purified ACF, NAP-1, topoisomerase I, plasmid DNA, and ATP in the presence or absence of purified core histones. The reaction products were subjected to micrococcal nuclease digestion analysis.

FIG. 4B shows the results of strand pairing analysis performed with the samples generated in FIG. 4A. Purified Drosophila Rad51 and Rad54 polypeptides were used in conjunction with the DL2 oligonucleotide. D loop reactions were performed as in FIG. 3B, except that the final concentration of Rad54 was 27 nM. The effect of non-nucleosomal histones upon strand pairing was also tested by the addition of core histones (the same amount as that used in the center lane) to the DNA after mock chromatin assembly (with ACF, NAP-1, DNA, ATP, and topoisomerase I in the absence of core histones) and immediately prior to the strand pairing reactions (right lane).). In contrast, the addition of core histones after a mock assembly reaction (carried out in the absence of histones) did not stimulate D loop formation (FIG. 3B, right lane). These results indicate that strand pairing is enhanced by chromatin (i.e., nucleosomal polynucleotides) but not non-nucleosomal core histones.

The data provided in FIG. 5 indicate that Rad54 and Rad51, or activities associated therewith, function cooperatively in the remodeling of chromatin. Restriction enzyme accessibility assays were carried out with naked DNA or chromatin, the indicated factors, and the restriction enzyme Hae III (15 units, Gibco-BRL) in the same reaction medium used for D loop reactions. The reactions were incubated for 1 hour at 27° C. The samples were deproteinized and subjected to electrophoresis on a 1% agarose gel. The DNA was visualized by staining with ethidium bromide. The final concentrations of the components, which were included as indicated, were as follows: plasmid DNA or chromatin (2 nM), DL2 oligonucleotide (1 nM), ATP (2 mM), Rad51 (200 nM), Rad54 (46 nM), ACF (3 nM). The amount of remodeling observed increases with the concentration of the factors (Rad51 and Rad54) as well as with the reaction time.

The foregoing description, for purposes of explanation, used specific nomenclature to provide a thorough understanding of the invention. Nevertheless, the foregoing descriptions of the preferred embodiments of the present invention are presented for purposes of illustration and description and are not intended to be exhaustive or to limit the invention to the precise forms disclosed; obvious modifications and variations are possible in view of the above teachings. Accordingly, it is intended that the scope of the invention be defined by the following claims.

1. A method for promoting homologous recombination, the method comprising providing a nucleosomal polynucleotide comprising histones and contacting, under conditions that support homologous recombination, the polynucleotide with a target nucleic acid sequence, wherein the target nucleic acid comprises a nucleotide sequence homologous to the nucleosomal polynucleotide.

2. The method of claim 1, wherein the conditions that support homologous recombination include a recombinase.

3. The method of claim 2, wherein the recombinase comprises Rad51 associated activity.

4. The method of claim 2, wherein the recombinase comprises Rad54 associated activity.

5. The method of claim 2, wherein the recombinase is exogenously produced.

6. The method of claim 2, wherein the recombinase is endogenously produced.

7. The method of claim 2, wherein the recombinase is a recombinosome.

8. The method of claim 1, wherein the contacting is in vitro.

9. The method of claim 1, wherein the contacting is in vivo.

10. The method of claim 1, wherein the target nucleic acid sequence is an exogenous sequence.

11. The method of claim 1, wherein the target nucleic acid sequence is an endogenous sequence.

12. The method of claim 11, wherein the endogenous sequence is a chromosomal sequence.

13. The method of claim 1, wherein the target nucleic acid sequence is coding sequence.

14. The method of claim 1, wherein the target nucleic acid sequence is non-coding sequence.

15. The method of claim 14, wherein the non-coding sequence is a promoter, enhancers, silencer, origin of replication or splicing signal sequence.

16. The method of claim 1, wherein the histones are core histones.

17. The method of claim 1, wherein the nucleosomal polynucleotide is a plasmid.

18. The method of claim 1, wherein the nucleosomal polynucleotide comprises a nucleic acid sequence that corrects a genetic mutation associated with a disease allele.

19. The method of claim 1, wherein the nucleosomal polynucleotide comprises a nucleic acid sequence that generates a genetic mutation in a targeted sequence.

20. The method of claim 18, wherein the genetic mutation is selected from the group consisting of base substitutions, additions, and deletions, or any combination thereof.

21. The method of claim 19, wherein the genetic mutation alters the expression of one or more genes in a targeted nucleic acid sequence.

22. A method of ameliorating disease caused by a disease allele, the method comprising:
a) providing a nucleosomal polynucleotide comprising histones and a nucleic acid sequence that corrects a genetic mutation associated with a disease allele; and
b) contacting, under conditions that support homologous recombination, the polynucleotide of a) with a target nucleic acid sequence associated with the disease allele, wherein the target nucleic acid comprises a nucleotide sequence homologous to the nucleosomal polynucleotide.

23. The method of claim 22, wherein the contacting is in vivo.

24. The method of claim 22, wherein the conditions that support homologous recombination include a recombinase.

25. The method of claim 24, wherein the recombinase comprises Rad51 and Rad54 associated activity.

26. The method of claim 24, wherein the recombinase is endogenously produced.

27. The method of claim 22, wherein the contacting is in vivo.

28. The method of claim 22, wherein the target nucleic acid sequence is an endogenous sequence.

29. The method of claim 28, wherein the endogenous sequence is a chromosomal sequence.

30. A method for promoting homologous strand pairing, the method comprising providing a nucleosomal polynucleotide comprising core histones and contacting, under conditions that support homologous strand pairing, the polynucleotide with a target nucleic acid sequence comprising a sequence homologous to the polynucleotide.

31. The method of claim 19, wherein the genetic mutation is selected from the group consisting of base substitutions, additions, and deletions, or any combination thereof.
